(19) United States
(12) Patent Application Publication
(10) Pub. No.: US 2021/0222111 A1
(43) Pub. Date: Jul. 22, 2021

(54) BIOREACTOR WASTE HEAT UTILIZATION

(71) Applicant: ExxonMobil Research and Engineering Company, Annandale, NJ (US)

(72) Inventors: Everett J. O'Neal, Asbury, NJ (US); Kevin B. Daly, Jersey City, NJ (US)

(21) Appl. No.: 17/082,244

(22) Filed: Oct. 28, 2020

Related U.S. Application Data
(60) Provisional application No. 62/963,278, filed on Jan. 20, 2020.

(51) Int. Cl. C12M 1/02; C12M 1/00; C12N 1/12

(52) U.S. Cl. CPC C12M 41/18; C12M 21/02; C12M 43/08; C12N 1/12; C12M 41/14; C12M 23/18

(57) ABSTRACT

A method of operating a bioreactor includes containing an algae slurry within the bioreactor for cultivation, discharging a portion of the algae slurry to a heat pump that circulates a refrigerant, and receiving the portion of the algae slurry at a first heat exchanger and transferring heat from the algae slurry to the refrigerant. The method further includes discharging a cooled algae slurry and a heated refrigerant from the first heat exchanger, receiving and compressing the heated refrigerant at a compressor and thereby discharging a compressed refrigerant, and receiving the compressed refrigerant at a second heat exchanger and transferring heat from the compressed refrigerant to a fluid. The method further includes discharging a cooled refrigerant and steam from the second heat exchanger, receiving and expanding the cooled refrigerant at an expansion valve, and receiving and utilizing the steam at a downstream application in fluid communication with the second heat exchanger.

### CROSS-REFERENCE TO RELATED APPLICATIONS

This application claims the benefit of priority from U.S. Provisional Application No. 62/963,278 filed Jan. 20, 2020, which is herein incorporated by reference in its entirety.

### FIELD OF THE INVENTION

The present disclosure is related to algal biomass cultivation and processing and, more particularly, to systems and methods for removing waste heat from bioreactors and utilizing the waste heat to generate steam useful for downstream applications.

### BACKGROUND OF THE INVENTION

Concerns about climate change, carbon dioxide (CO₂) emissions, and depleting mineral oil and gas resources have led to widespread interest in the production of biofuels from algae, including microalgae. As compared to other plant-based feedstocks, algae have higher CO₂ fixation efficiencies and growth rates, and growing algae can efficiently utilize wastewater, biomass residue, and industrial gases as nutrient sources.

Algae are photoautotrophic organisms that can survive, grow, and reproduce with energy derived entirely from the sun through the process of photosynthesis. Photosynthesis is essentially a carbon recycling process through which inorganic CO₂ combines with solar energy, other nutrients, and cellular biochemical processes to output gaseous oxygen and to synthesize carbohydrates and other compounds critical to the life of the algae.

To produce algal biomass, algae cells are generally grown in a water slurry comprising water and nutrients. The algae may be cultivated in indoor or outdoor environments, and in closed or open cultivation systems or “bioreactors.” Closed bioreactors are commonly referred to as “photobioreactors” and utilize natural or artificial light to grow algae in an environment that is generally isolated from the external atmosphere. Such photobioreactors may be in a variety of shaped configurations, but are typically tubular or flat paneled. Open bioreactors include natural and artificial ponds that utilize sunlight to facilitate photosynthesis. Artificial ponds are often shaped in circular or raceway-shaped (oval) configurations (referred to as “raceway ponds”).

Various processing methods exist for extracting lipids (oils) from harvested biomass for the production of fuel and other oil-based products. One traditional method of extracting lipids includes processing an algal biomass into a paste and then drying the paste to a moisture level of about 10% or less. The biomass is then further processed in an extruder or other mechanical shearing device to lyse the algae cells. Various chemicals (e.g., hexane) are used to extract the lipids from the lysed algae cells for use in biofuel production.

Drying the algal biomass prior to lipid extraction requires a substantial amount of energy to reduce the moisture of the algal biomass to acceptable levels. Steam is often used as the source of energy to help dry the algal biomass, and what is needed is an energy-efficient means of generating and capturing steam to help aid algal biomass drying processes.

### SUMMARY OF THE INVENTION

The present disclosure is related to algal biomass cultivation and processing and, more particularly, to systems and methods for removing waste heat from bioreactors and utilizing the waste heat to generate steam useful for downstream applications.

In some aspects, a system is disclosed that includes a bioreactor to contain an algae slurry for cultivation, a heat pump that circulates a refrigerant and is in fluid communication with the bioreactor, the heat pump including a first heat exchanger that receives a portion of the algae slurry from the bioreactor and transfers heat from the portion of the algae slurry to the refrigerant, whereby a cooled algae slurry and a heated refrigerant are discharged from the first heat exchanger, a compressor that receives and compresses the heated refrigerant and discharges a compressed refrigerant, a second heat exchanger that receives the compressed refrigerant and transfers heat from the compressed refrigerant to a fluid, whereby a cooled refrigerant and steam are discharged from the second heat exchanger, and an expansion valve that receives and expands the cooled refrigerant. The system may further include a downstream application in fluid communication with the second heat exchanger to receive and utilize the steam.

In some aspects, a method is disclosed that includes containing an algae slurry within the bioreactor for cultivation, discharging a portion of the algae slurry to a heat pump that circulates a refrigerant, receiving the portion of the algae slurry at a first heat exchanger of the heat pump and transferring heat from the portion of the algae slurry to the refrigerant, discharging a cooled algae slurry and a heated refrigerant from the first heat exchanger, receiving and compressing the heated refrigerant at a compressor of the heat pump and thereby discharging a compressed refrigerant, receiving the compressed refrigerant at a second heat exchanger of the heat pump and transferring heat from the compressed refrigerant to a fluid, discharging a cooled refrigerant and steam from the second heat exchanger, receiving and expanding the cooled refrigerant at an expansion valve of the heat pump, and receiving and utilizing the steam at a downstream application in fluid communication with the second heat exchanger.

### BRIEF DESCRIPTION OF THE DRAWINGS

The following figures are included to illustrate certain aspects of the present disclosure, and should not be viewed as exclusive examples. The subject matter disclosed is capable of considerable modifications, alterations, combinations, and equivalents in form and function, without departing from the scope of this disclosure.

FIG. 1 is an example system that may be used to grow and harvest algae for biofuel production.

FIG. 2 is a schematic diagram of the heat pump of FIG. 1 used in conjunction with the photobioreactor of FIG. 1, according to one or more aspects of the present disclosure.

### DETAILED DESCRIPTION OF THE INVENTION

Biofuel production from harvested algal biomass offers sustainable energy solutions to reduce reliance on fossil fuels and reduce greenhouse gas emissions. To accomplish substantial economic, environmental, and societal impact, algae is typically cultivated in large-scale systems to produce large quantities of algal biomass. Such large-scale cultivation systems allow algae-derived biofuels to become more cost-effective and more widely available to the public. Because of the substantial amount of drying required to achieve a desired moisture content, the biomass drying process is often a major bottleneck in terms of production costs, energy costs, time, and environmental impact. Steam is often utilized as an energy source to aid algal biomass drying, and drying algae on a 10 kbd scale, for example, can require large steam requirements; e.g., 70 MW-200 MW, as per commercial design calculations. Although this is a large amount of steam, the temperature at which the steam could be raised may be as low as 90° C. (sub-atmospheric) for moisture removal.

In high-temperature algae growing environments, bioreactors accumulate heat during the day which is normally rejected to the surrounding environment or atmosphere. In some cases, cooling water is used to draw heat from the bioreactor, but the cooling water must be clean and desalinated, which affects the system's ability to function with a low environmental footprint. According to various aspects of the present disclosure, a heat pump may be employed to convert low-value waste heat accumulated in bioreactors to low-pressure steam. As the heat pump cools the algae slurry solution, the waste heat drawn from the algae slurry is utilized to generate steam that can be used for a variety of purposes, such as algal biomass drying, direct air capture systems, amine capture systems, power generation, or other applications that require steam.

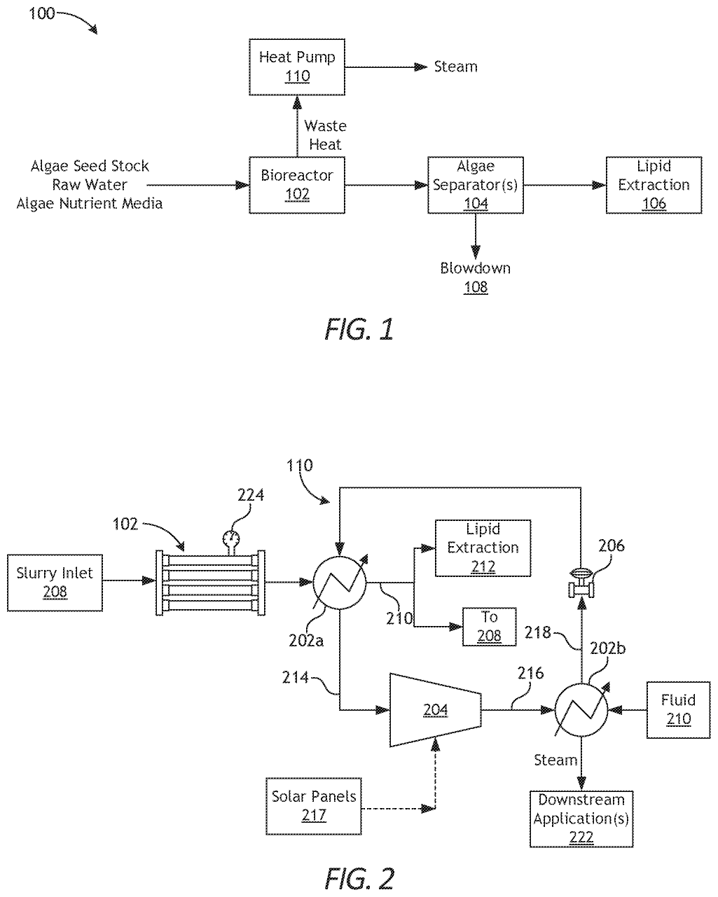

FIG. 1 is an example system 100 that may be used to grow and harvest algae for biofuel production. As illustrated, the system 100 includes a bioreactor 102. As used herein, the term “bioreactor” refers generally to any open or closed algae cultivation vessel or system used for the growth of algal biomass, including closed-system photobioreactors, natural ponds, artificial ponds (e.g., raceway ponds), and the like, and including any combinations thereof. The principles of the present disclosure are preferably used in conjunction with tubular-type, closed algae cultivation vessels/systems or “photobioreactors,” but are equally applicable to open algae cultivation vessels/systems.

The bioreactor 102 may be fed with raw water, an algae feedstock, and algae nutrient media to help create and contain an algae slurry for cultivation and growth. As used herein, the term “algae slurry,” and grammatical variants thereof, refers to a flowable liquid comprising at least water, algae cells, and algae nutrient media.

Algal sources for preparing the algae slurry include, but are not limited to, unicellular and multicellular algae. Examples of such algae can include, but are not limited to, a rhodophyte, chlorophyte, heterokontophyte, tribophyte, glaucophyte, chlorarachniophyte, euglenoid, haptophyte, cryptomonad, dinoflagellum, phytoplankton, and the like, and combinations thereof. In some examples, algae can be of the classes Chlorophyceae and/or Haptophyta. Specific species can include, but are not limited to, Neochloris oleoabundans, Scenedesmus dimorphus, Euglena gracilis, Phaeodactylum tricornutum, Pleurochrysis carterae, Prymnesium parvum, Tetraselmis chui, and Chlamydomonas reinhardtii. Additional or alternate algal sources can include one or more microalgae of the Achnanthes, Amphiprora, Amphora, Ankistrodesmus, Asteromonas, Boekelovia, Borodinella, Botryococcus, Bracteococcus, Chaetoceros, Carteria, Chlamydomonas, Chlorococcum, Chlorogonium, Chlorella, Chroomonas, Chrysosphaera, Cricosphaera, Crypthecodinium, Cryptomonas, Cyclotella, Dunaliella, Ellipsoidon, Emiliania, Eremosphaera, Ernodesmius, Euglena, Franceia, Fragilaria, Gloeothamnion, Haematococcus, Halocafeteria, Hymenomonas, Isochrysis, Lepocinclis, Micractinium, Monoraphidium, Nannochloris, Nannochloropsis, Navicula, Neochloris, Nephrochloris, Nephroselmis, Nitzschia, Ochromonas, Oedogonium, Oocystis, Ostreococcus, Pavlova, Parachlorella, Pascheria, Phaeodactylum, Phagus, Pichochlorum, Pseudoneochloris, Pseudostaurastrum, Platymonas, Pleurochrysis, Pleurococcus, Prototheca, Pseudochlorella, Pyramimonas, Pyrobotrys, Scenedesmus, Schizochlamydella, Skeletonema, Spyrogyra, Stichococcus, Tetrachlorella, Tetraselmis, Thalassiosira, Tribonema, Vaucheria, Viridiella, and Volvox species, and/or one or more cyanobacteria of the Agmenellum, Anabaena, Anabaenopsis, Anacystis, Aphanizomenon, Arthrospira, Asterocapsa, Borzia, Calothrix, Chamaesiphon, Chlorogloeopsis, Chroococcidiopsis, Chroococcus, Crinalium, Cyanobacterium, Cyanobium, Cyanocystis, Cyanospira, Cyanothece, Cylindrospermopsis, Cylindrospermum, Dactylococcopsis, Dermocarpella, Fischerella, Fremyella, Geitleria, Geitlerinema, Gloeobacter, Gloeocapsa, Gloeothece, Halospirulina, Iyengariella, Leptolyngbya, Limnothrix, Lyngbya, Microcoleus, Microcystis, Myxosarcina, Nodularia, Nostoc, Nostochopsis, Oscillatoria, Phormidium, Planktothrix, Pleurocapsa, Prochlorococcus, Prochloron, Prochlorothrix, Pseudanabaena, Rivularia, Schizothrix, Scytonema, Spirulina, Stanieria, Starria, Stigonema, Symploca, Synechococcus, Synechocystis, Tolypothrix, Trichodesmium, Tychonema, and Xenococcus species. Any combination of the aforementioned algae sources may additionally be used to prepare an algae slurry.

The raw water used in preparing the algae slurry may originate from any water source including, but not limited to, fresh water, brackish water, seawater, synthetic seawater (e.g., water with added salts), wastewater (treated or untreated), or any combination thereof.

The algae nutrient media used in cultivating the algae slurry may comprise at least nitrogen (e.g., in the form of ammonium nitrate or ammonium urea) and phosphorous. Other elemental micronutrients may also be included, such as potassium, iron, manganese, copper, zinc, molybdenum, vanadium, boron, chloride, cobalt, silicon, and the like, and any combination thereof.

During typical operation, the algae slurry resides in the bioreactor 102 for a predetermined amount of time or until the algae matures and is ready for harvesting. Typical residence time in the bioreactor 102 can range between about 2 days and about 20 days. Once the algae matures and is otherwise ready for harvesting, the algae slurry is discharged from the bioreactor 102 and pumped to one or more algae-water separators 104 to be dewatered, during which process the algae in the algae slurry is generally separated from the water. The algae-water separator(s) 104 may comprise any known separator, filter, or dewatering system known, and can include any combination thereof.

The separated algae is then conveyed downstream for lipid extraction 106 in preparation for biofuel production or the creation of other oil-based products. Harvesting cultivated algal biomass can alternatively be used to produce non-fuel or non-oil-based products, including nutraceuticals, pharmaceuticals, cosmetics, chemicals (e.g., paints, dyes, and colorants), fertilizer and animal feed, and the like. Various processing methods exist for harvesting cultivated algal biomass to extract lipids therefrom. Such methods traditionally include the addition of one or more chemicals or the use of mechanical equipment to physically separate algae from the remaining components of a water slurry.

The separated water can be purged from the system 100 via a blowdown stream 108 and discharged into the environment or reused for another application. In some cases, the separated water purged via the blowdown stream 108 is conveyed to a wastewater treatment plant for treatment so that the separated water can be discharged into the environment with minimal impact.

One challenge confronted in algae cultivation is maintaining the bioreactor 102 at a temperature suitable for the cultivation and growth of various algae strains. The bioreactor 102 will continuously accumulate thermal energy (heat) during the day as it is directly exposed to the sun. If the temperature of the algae slurry exceeds a predetermined upper temperature limit (e.g., about 40° C.), some algae strains will begin to die. The bioreactor 102 will typically be cooled periodically during the day to remove heat from the algae slurry and thereby maintain the temperature below the predetermined upper temperature limit. In applications where the bioreactor 102 is a closed-tube photobioreactor, cooling the bioreactor 102 is often done by exposing the external surfaces of the bioreactor 102 to clean, desalinated water, which draws heat from the algae slurry as the water evaporates. The heat drawn from the algae slurry is consequently released into the atmosphere or surrounding environment as waste heat.

According to various aspects of the present disclosure, a heat pump 110 and associated thermodynamic cycle may be incorporated into the system 100 to receive and utilize the waste heat emitted from the bioreactor 102 to generate steam. Instead of losing the waste heat to the atmosphere or surrounding environment as lost thermal energy, the heat pump 110 utilizes the waste heat to generate steam usable for a variety of valuable purposes including, but not limited to, algal biomass drying, direct air capture systems, an amine capture plant, power generation, or other applications that require steam.

FIG. 2 is a schematic diagram of the heat pump 110 of FIG. 1 used in conjunction with the bioreactor 102, according to one or more aspects of the present disclosure. As illustrated, the bioreactor 102 is in the form of a closed-tube photobioreactor, but could alternatively be any type of bioreactor mentioned herein, without departing from the scope of the disclosure. The heat pump 110 includes a first heat exchanger or “evaporator” 202a, a compressor 204, a second heat exchanger or “condenser” 202b, and an expansion valve 206. The heat exchangers 202a,b, the compressor 204, and the expansion valve 206 are all fluidly interconnected using suitable piping, conduits, and connectors to form a continuous thermodynamic cycle capable of circulating a heat transfer medium or “refrigerant.”

In some applications, the refrigerant circulated through the heat pump 110 may comprise butane, but may alternatively comprise other types of refrigerants, without departing from the scope of the disclosure. Example refrigerants that may be used in accordance with the principles of the present disclosure include, but are not limited to ethane, ethylene, propane, propylene, isobutene, 1-butylene, 2-butylene, pentane, isopentane, ammonia, methylamine, ethylamine, methyl formate or other refrigerants.

The heat exchangers 202a,b may comprise any type of heat exchanging device, apparatus, or system capable of increasing or decreasing the temperature of the refrigerant circulating through the heat pump 110. The first heat exchanger 202a, for example, may be configured to increase the temperature of the refrigerant, and the second heat exchanger 202b may be configured to decrease the temperature of the refrigerant, as described in more detail below. Examples of the heat exchangers 202a,b include, but are not limited to, a shell and tube heat exchanger, a plate heat exchanger, a plate and shell heat exchanger, an adiabatic wheel heat exchanger, a plate fin heat exchanger, a pillow plate heat exchanger, a fluid heat exchanger, a helical-coil heat exchanger, a spiral heat exchanger, a direct gas/liquid contact system, or any combination thereof.

Example operation of the heat pump 110 in conjunction with the bioreactor 102 is now provided. An algae slurry can be introduced into the bioreactor 102 at a slurry inlet 208. While contained within the bioreactor 102, the algae slurry may be heated as it is exposed to the sun during the daylight hours and the temperature of the bioreactor 102 may reach or exceed a predetermined upper temperature limit. The predetermined upper temperature limit may vary depending on the size of the bioreactor 102 and the type of algae strain included in the algae slurry. In some embodiments, the predetermined upper temperature limit may range between about 40° C. and about 45° C., above which point some algae strains are unable to survive.

To cool the bioreactor 102, all or a portion of the algae slurry may be discharged from the bioreactor 102 and conveyed to the first heat exchanger 202a. The first heat exchanger 202a operates to reduce the temperature of the algae slurry and thereby discharge a cooled algae slurry 210. In some embodiments, the first heat exchanger 202 may be capable of cooling the algae slurry from at or near the predetermined upper temperature limit to about 25 to 35° C. Depending on its maturity, the cooled algae slurry 210 may either be returned to the slurry inlet 208 for further cultivation and growth within the bioreactor 102, or may be conveyed downstream for harvesting and lipid extraction 212.

The refrigerant is introduced into the first heat exchanger 202a to draw thermal energy from the algae slurry, thus capturing the waste heat from the bioreactor and resulting in the cooled algae slurry 210 discharged from the first heat exchanger 202a. In some embodiments, at least a portion of the refrigerant may make direct contact with the algae slurry to both cool and provide carbon or nitrogen to the algae slurry. In such embodiments, the refrigerant will advantageously help cool the algae slurry, but also provide valuable nutrients for growth.

In embodiments where the refrigerant is butane, the refrigerant may be introduced into the first heat exchanger 202a at a temperature of about 0 to 25° C. As the refrigerant circulates through the first heat exchanger 202a, heat is drawn from the algae slurry to the refrigerant, which evaporates the refrigerant such that the first heat exchanger 202a discharges a gaseous, heated refrigerant 214. In some embodiments, the heated refrigerant 214 may be discharged from the first heat exchanger 202a at a temperature ranging between about 30° C. and about 40° C., encompassing any value and subset there between.

The heated refrigerant 214 may then be conveyed to the compressor 204, which compresses the gaseous, heated refrigerant 214 and discharges a compressed refrigerant 216 exhibiting an increased temperature. In some embodiments, the compressed refrigerant 216 may exhibit a temperature ranging between about 80° C. and about 150° C., encompassing any value and subset there between. In such embodiments, the compressor 204 may require around 35 MW of electricity to properly compress the heated refrigerant 214. In some embodiments, the compressor 204 may be powered using electricity derived at least in part from photovoltaic solar panels 217 included in the system 100 (FIG. 1), thus increasing the efficiency of the heat pump 110 and the system 100 as a whole.

The compressed refrigerant 216 is then conveyed to the second heat exchanger 202b, which operates to decrease the temperature of the compressed refrigerant 216 and discharge a cooled refrigerant 218. In some embodiments, the cooled refrigerant 218 may be discharged from the second heat exchanger 202b at a temperature ranging between about 50° C. and about 140° C., encompassing any value and subset there between. To cool the compressed refrigerant 216, the second heat exchanger 202b also receives a fluid 220 that exchanges thermal energy (heat) with the compressed refrigerant 216. In some embodiments, the fluid 220 may comprise water in the form of steam, a mixture of steam and liquid water, or liquid water. In at least one embodiment, however, the fluid 220 may alternatively comprise air. As the fluid 220 circulates through the second heat exchanger 202b, heat is drawn from the compressed refrigerant 216 to the fluid 220, thus resulting in the cooled refrigerant 218.

The heat drawn from the compressed refrigerant 216 serves to convert the fluid 220 into steam at low pressure (e.g., 0.8 bar), or alternatively into hot air. In some applications, the generated steam (or hot air) may provide around 150 MW of heating value, which can be used for a variety of downstream applications 222. As mentioned herein, one downstream application 222 includes using the steam to help dry algal biomass, thus helping to solve the energy load issue associated with algae drying. In such embodiments, the steam is conveyed to one or more rotating drum dryers (not shown), and the algae slurry is flowed over an outside surface of the drum dryer(s), which dries the algae slurry into a paste or flake material. Swept air (e.g., heated air derived from a greenhouse-enclosed solar steam system or other application) may further be flowed across the algae slurry as it is treated on the drum dryer(s) to increase drying effectiveness and efficiency.

Another downstream application 222 includes using the steam in a direct air capture system to help capture carbon-dioxide (CO₂). In such embodiments, the direct air capture system may include large contacting beds through which air is flowed and the CO₂ in the air adheres to the contacting beds. The thermal energy from the steam may be used to regenerate the contacting beds and thereby remove the CO₂. The steam may alternatively be used for end-use regenerating an amine capture plant. In such embodiments, the amine capture plant will capture the CO₂, and the steam will be used to regenerate those amines.

In yet another downstream application 222, the steam may be used to generate electricity usable to power a variety of other processes or devices. More specifically, the steam may be conveyed to a turbine generator and act as a working fluid that drives the turbine in rotation, which results in electricity generation.

Still referring to FIG. 2, the cooled refrigerant 218 may be conveyed to and throttled (expanded) across the expansion valve 206, which decreases the temperature and the pressure of the refrigerant back to or near the initial temperature and pressure of the refrigerant prior to being introduced into the first heat exchanger 202a. At this point, the process can start over as the refrigerant is once again circuited through the heat pump 110 to remove heat from the algae slurry discharged from the bioreactor 102. In some embodiments, the heat pump 110 is operated at least once a day to remove heat from the bioreactor 102. In at least one embodiment, the temperature of the algae slurry may be monitored continuously using a temperature gauge or sensor 224, and the heat pump 110 may be operated at any time the temperature of the algae slurry approaches or reaches the predetermined upper temperature limit.

As indicated above, a variety of refrigerants may be used in the heat pump 110, without departing from the scope of the disclosure. Suitable refrigerants will have fluid properties such that the first heat exchanger 202a is able to increase the temperature of the refrigerant to a temperature ranging between about 30° C. and about 40° C., the compressor 204 is able to increase the temperature of the refrigerant to 100° C. or more, and the expansion valve 206 is able to reduce the temperature of the refrigerant to less than 30° C.

Those skilled in the art will readily appreciate that using a heat pump 110 in conjunction with algae photobioreactors 102 is not conventional. Incorporating the heat pump 110, however, allows operators to design the bioreactor 110 to benefit the heat pump 110 application. For example, incorporating the heat pump 110 allows the bioreactor 110 to be designed with smaller diameter tubes, which are commonly ruled out because they overheat too quickly. With the heat pump 110, however, thermal energy can be removed from the bioreactor 102 continuously or as needed, thus maintaining the temperature of the bioreactor 102 at suitable levels for algae growth.

### Embodiments Listing

The present disclosure provides, among others, the following examples, each of which may be considered as optionally including any alternate example.

Clause 1. A system includes a bioreactor to contain an algae slurry for cultivation, a heat pump that circulates a refrigerant and is in fluid communication with the bioreactor, the heat pump including a first heat exchanger that receives a portion of the algae slurry from the bioreactor and transfers heat from the portion of the algae slurry to the refrigerant, whereby a cooled algae slurry and a heated refrigerant are discharged from the first heat exchanger, a compressor that receives and compresses the heated refrigerant and discharges a compressed refrigerant, a second heat exchanger that receives the compressed refrigerant and transfers heat from the compressed refrigerant to a fluid, whereby a cooled refrigerant and steam are discharged from the second heat exchanger, and an expansion valve that receives and expands the cooled refrigerant. The system further including a downstream application in fluid communication with the second heat exchanger to receive and utilize the steam.

Clause 2. The system of Clause 1, wherein the bioreactor is selected from the group consisting of a closed-system photobioreactor, a natural pond, an artificial pond, and any combination thereof.

Clause 3. The system of Clause 1 or 2, wherein the refrigerant is selected from the group consisting of butane, ethane, ethylene, propane, propylene, isobutene, 1-butylene, 2-butylene, pentane, isopentane, ammonia, methylamine, ethylamine, methyl formate, and any combination thereof.

Clause 4. The system of any of the preceding Clauses, wherein the first and second heat exchangers are selected from the group consisting of a shell and tube heat exchanger, a plate heat exchanger, a plate and shell heat exchanger, an adiabatic wheel heat exchanger, a plate fin heat exchanger, a pillow plate heat exchanger, a fluid heat exchanger, a helical-coil heat exchanger, a spiral heat exchanger, a direct gas/liquid contact system, and any combination thereof.

Clause 5. The system of any of the preceding Clauses, wherein the cooled algae slurry is returned to the bioreactor.

Clause 6. The system of any of Clauses 1 through 4, wherein the cooled algae slurry is processed for lipid extraction.

Clause 7. The system of any of the preceding Clauses, wherein the compressed refrigerant exhibits a temperature greater than the heated refrigerant.

Clause 8. The system of any of the preceding Clauses, wherein the fluid is selected from the group consisting of steam, liquid water, a mixture of steam and liquid water, and air.

Clause 9. The system of any of the preceding Clauses, wherein the downstream application comprises at least one of an algal biomass drying application, a direct air capture system, an amine capture plant, and a turbine that generates electricity.

Clause 10. The system of any of the preceding Clauses, further comprising one or more photovoltaic solar panels that generate electricity to power the compressor.

Clause 11. A method of operating a bioreactor, the method including containing an algae slurry within the bioreactor for cultivation, discharging a portion of the algae slurry to a heat pump that circulates a refrigerant, receiving the portion of the algae slurry at a first heat exchanger of the heat pump and transferring heat from the portion of the algae slurry to the refrigerant, discharging a cooled algae slurry and a heated refrigerant from the first heat exchanger, receiving and compressing the heated refrigerant at a compressor of the heat pump and thereby discharging a compressed refrigerant, receiving the compressed refrigerant at a second heat exchanger of the heat pump and transferring heat from the compressed refrigerant to a fluid, discharging a cooled refrigerant and steam from the second heat exchanger, receiving and expanding the cooled refrigerant at an expansion valve of the heat pump, and receiving and utilizing the steam at a downstream application in fluid communication with the second heat exchanger.

Clause 12. The method of Clause 11, further comprising returning the cooled algae slurry to the bioreactor.

Clause 13. The method of Clause 11, further comprising processing the cooled algae slurry for lipid extraction.

Clause 14. The method of any of Clauses 11 through 13, wherein receiving and compressing the heated refrigerant at the compressor comprises increasing a temperature of the heated refrigerant.

Clause 15. The method of any of Clauses 11 through 14, wherein the downstream application comprises an algal biomass drying application, the method further comprising conveying the steam to a rotating drum dryer, flowing the algae slurry over an outside surface of the drum dryer, and drying the algae slurry into a paste or flake material.

Clause 16. The method of any of Clauses 11 through 14, wherein the downstream application comprises a direct air capture system, the method further comprising conveying the steam to one or more contacting beds of the direct air capture system, and contacting the steam on the one or more contacting beds and thereby removing carbon dioxide adhered to the one or more contacting beds.

Clause 17. The method of any of Clauses 11 through 14, wherein the downstream application comprises a turbine generator, the method further comprising conveying the steam to a turbine generator, and generating electricity as the steam rotates the turbine generator.

Clause 18. The method of any of Clauses 11 through 17, further comprising monitoring a temperature of the algae slurry within the bioreactor, and discharging the portion of the algae slurry from the bioreactor when the temperature of the algae slurry reaches or exceeds a predetermined upper temperature limit.

Clause 19. The method of any of Clauses 11 through 17, further comprising powering the compressor with electricity derived at least in part from photovoltaic solar panels.

Clause 20. The method of any of Clauses 11 through 19, wherein transferring heat from the portion of the algae slurry to the refrigerant comprises directly contacting at least a portion of the refrigerant with the algae slurry, and providing nutrients to the algae slurry from the refrigerant.

One or more illustrative incarnations incorporating one or more invention elements are presented herein. Not all features of a physical implementation are described or shown in this application for the sake of clarity. It is understood that in the development of a physical embodiment incorporating one or more elements of the present invention, numerous implementation-specific decisions must be made to achieve the developer's goals, such as compliance with system-related, business-related, government-related and other constraints, which vary by implementation and from time to time. While a developer's efforts might be time-consuming, such efforts would be, nevertheless, a routine undertaking for those of ordinary skill in the art and having benefit of this disclosure.

While compositions and methods are described herein in terms of “comprising” various components or steps, the compositions and methods can also “consist essentially of” or “consist of” the various components and steps.

Therefore, the present invention is well adapted to attain the ends and advantages mentioned as well as those that are inherent therein. The particular examples and configurations disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative examples disclosed above may be altered, combined, or modified and all such variations are considered within the scope and spirit of the present invention. The invention illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces.

What is claimed is:

1. A system, comprising:
a bioreactor to contain an algae slurry for cultivation;
a heat pump that circulates a refrigerant and is in fluid communication with the bioreactor, the heat pump including:
a first heat exchanger that receives a portion of the algae slurry from the bioreactor and transfers heat from the portion of the algae slurry to the refrigerant, whereby a cooled algae slurry and a heated refrigerant are discharged from the first heat exchanger;
a compressor that receives and compresses the heated refrigerant and discharges a compressed refrigerant;
a second heat exchanger that receives the compressed refrigerant and transfers heat from the compressed refrigerant to a fluid, whereby a cooled refrigerant and steam are discharged from the second heat exchanger; and
an expansion valve that receives and expands the cooled refrigerant; and
a downstream application in fluid communication with the second heat exchanger to receive and utilize the steam.

2. The system of claim 1, wherein the bioreactor is selected from the group consisting of a closed-system photobioreactor, a natural pond, an artificial pond, and any combination thereof.

3. The system of claim 1, wherein the refrigerant is selected from the group consisting of butane, ethane, ethylene, propane, propylene, isobutene, 1-butylene, 2-butylene, pentane, isopentane, ammonia, methylamine, ethylamine, methyl formate, and any combination thereof.

4. The system of claim 1, wherein the first and second heat exchangers are selected from the group consisting of a shell and tube heat exchanger, a plate heat exchanger, a plate and shell heat exchanger, an adiabatic wheel heat exchanger, a plate fin heat exchanger, a pillow plate heat exchanger, a fluid heat exchanger, a helical-coil heat exchanger, a spiral heat exchanger, a direct gas/liquid contact system, and any combination thereof.

5. The system of claim 1, wherein the cooled algae slurry is returned to the bioreactor.

6. The system of claim 1, wherein the cooled algae slurry is processed for lipid extraction.

7. The system of claim 1, wherein the compressed refrigerant exhibits a temperature greater than the heated refrigerant.

8. The system of claim 1, wherein the fluid is selected from the group consisting of steam, liquid water, a mixture of steam and liquid water, and air.

9. The system of claim 1, wherein the downstream application comprises at least one of an algal biomass drying application, a direct air capture system, an amine capture plant, and a turbine that generates electricity.

10. The system of claim 1, further comprising one or more photovoltaic solar panels that generate electricity to power the compressor.

11. A method of operating a bioreactor, comprising:
containing an algae slurry within the bioreactor for cultivation;
discharging a portion of the algae slurry to a heat pump that circulates a refrigerant;
receiving the portion of the algae slurry at a first heat exchanger of the heat pump and transferring heat from the portion of the algae slurry to the refrigerant;
discharging a cooled algae slurry and a heated refrigerant from the first heat exchanger;
receiving and compressing the heated refrigerant at a compressor of the heat pump and thereby discharging a compressed refrigerant;
receiving the compressed refrigerant at a second heat exchanger of the heat pump and transferring heat from the compressed refrigerant to a fluid;
discharging a cooled refrigerant and steam from the second heat exchanger;
receiving and expanding the cooled refrigerant at an expansion valve of the heat pump; and
receiving and utilizing the steam at a downstream application in fluid communication with the second heat exchanger.

12. The method of claim 11, further comprising returning the cooled algae slurry to the bioreactor.

13. The method of claim 11, further comprising processing the cooled algae slurry for lipid extraction.

14. The method of claim 11, wherein receiving and compressing the heated refrigerant at the compressor comprises increasing a temperature of the heated refrigerant.

15. The method of claim 11, wherein the downstream application comprises an algal biomass drying application, the method further comprising:
conveying the steam to a rotating drum dryer;
flowing the algae slurry over an outside surface of the drum dryer; and
drying the algae slurry into a paste or flake material.

16. The method of claim 11, wherein the downstream application comprises a direct air capture system, the method further comprising:
conveying the steam to one or more contacting beds of the direct air capture system; and
contacting the steam on the one or more contacting beds and thereby removing carbon dioxide adhered to the one or more contacting beds.

17. The method of claim 11, wherein the downstream application comprises a turbine generator, the method further comprising:
conveying the steam to a turbine generator; and
generating electricity as the steam rotates the turbine generator.

18. The method of claim 11, further comprising:
monitoring a temperature of the algae slurry within the bioreactor; and
discharging the portion of the algae slurry from the bioreactor when the temperature of the algae slurry reaches or exceeds a predetermined upper temperature limit.

19. The method of claim 11, further comprising powering the compressor with electricity derived at least in part from photovoltaic solar panels.

20. The method of claim 11, wherein transferring heat from the portion of the algae slurry to the refrigerant comprises:
directly contacting at least a portion of the refrigerant with the algae slurry; and
providing nutrients to the algae slurry from the refrigerant.
